(19) United States
(12) Patent Application Publication
(10) Pub. No.: US 2021/0283034 A1
(43) Pub. Date: Sep. 16, 2021

(54) CLEANSING MATERIAL COMPOSITION

(71) Applicant: AJINOMOTO CO., INC., Tokyo (JP)

(72) Inventors: Naoaki IKEDA, Kawasaki-shi (JP); Shun KOBAYASHI, Yokkaichi-shi (JP)

(73) Assignee: AJINOMOTO CO., INC., Tokyo (JP)

(21) Appl. No.: 17/337,884

(22) Filed: Jun. 3, 2021

Related U.S. Application Data
(63) Continuation of application No. PCT/JP2019/047644, filed on Dec. 5, 2019.

(30) Foreign Application Priority Data
Dec. 7, 2018 (JP) 2018-229980

(51) Int. Cl. A61K 8/44; A61K 8/46; A61K 8/73; A61K 8/41; A61K 8/34; A61K 8/92; A61K 8/86; A61K 8/81; A61Q 19/10

(52) U.S. Cl. CPC A61K 8/44; A61K 8/466; A61K 8/732; A61K 8/416; A61K 8/345; A61K 2800/596; A61K 8/86; A61K 8/8152; A61Q 19/10; A61K 2800/594; A61K 8/922

(57) ABSTRACT

Cleansing material compositions which contain an acyl acidic amino acid or a salt thereof, an amphoteric surfactant, and hydroxypropyl starch phosphate, and have a pH of 5.3 or less are useful for facial cleansing.

### CROSS REFERENCES TO RELATED APPLICATIONS

This application is a continuation of International Patent Application No. PCT/JP2019/047644, filed on Dec. 5, 2019, and claims priority to Japanese Patent Application No. 2018-229980, filed on Dec. 7, 2018, both of which are incorporated herein by reference in their entireties.

### BACKGROUND OF THE INVENTION

### Field of the Invention

The present invention relates to cleansing material compositions

### Discussion of the Background

Commercial cream-type facial cleansing materials are roughly classified into crystallized types having surfactant crystals dispersed therein and thickened types having increased viscosity due to entanglement of surfactant molecule aggregates or polymers. The crystallized types have a high concentration of surfactants blended and tend to suffer an increase in the cost of raw materials in the product. The thickened types are thickened by entanglement among polymers and surfactant molecule aggregates called worm-like micelles. For this reason, the thickened types have advantages in that the concentration of the surfactants blended can be kept low and it is unnecessary to perform a complicated crystallization operation.

Meanwhile, as the surfactant, acyl acidic amino acids are known.

For example, Japanese Patent Application Publication No. 2004-155716 (Patent Literature 1, which is incorporated herein by reference in its entirety) discloses a cream-type skin cleansing material containing an acyl acidic amino acid, a sulfosuccinic acid-type surfactant, and a nonionic surfactant having an HLB of 10 or more for the purpose of providing a skin cleansing material which is mild to the skin, excellent in foaming power and foam quality, and excellent in usability.

Japanese Patent Application Publication No. 2008-100921 (Patent Literature 2, which is incorporated herein by reference in its entirety) discloses a facial cleansing material containing a thickening polymer compound having a specific structure, an acylamino acid-type surfactant at 1 to 30% by mass, and an alkylaminodicarboxylic acid-type surfactant as a technique relating to a cleansing material which is in a form for preventing excessive rubbing against the skin when washing the face, provides a refreshing feeling without giving the skin an unpleasant dry feeling, and imparts a smooth feeling to the skin after washing.

Japanese Patent Application Publication No. 2003-160797 (Patent Literature 3, which is incorporated herein by reference in its entirety) discloses a cleansing material which contains a specific anionic surfactant and a specific liquid ester oil in a specific ratio, contains a predetermined content of at least one or two selected from polyether-modified silicones and cationic polymers and a predetermined content of a predetermined dihydric alcohol, and contains substantially no antiseptic component.

Japanese Patent Application Publication No. 2008-88077 (Patent Literature 4, which is incorporated herein by reference in its entirety) discloses a hair conditioner which is a transparent gel-form composition containing (A) an N-long-chain acyl acidic amino acid and/or a salt thereof, (B) a cationic surfactant, and (C) water, wherein a weight ratio of the component (A) to the component (B) is in a predetermined range, and a content of the component (A) is in a predetermined range.

### SUMMARY OF THE INVENTION

Some cleansing material compositions are supplied in a tube-filled state, for example, such as face-washing foams. The tube-filled cleansing material composition is pushed out of the tube to the palm when used. Such composition is required to have viscosity to the extent that the composition does not flow down from the palm.

Although acyl acidic amino acids themselves have good foam performance, cleansing material compositions using them have difficulty in obtaining a viscosity to an extent which allows tube-filled use without impairing good foam performance.

Accordingly, it is one object of the present invention to provide a cleansing material composition using an acyl acidic amino acid, the cleansing material composition having a viscosity suitable for tube-filled use without impairing foam performance such as foam volume and foamability.

This and other objects, which will become apparent during the following detailed description, have been achieved by the following:

(1) A cleansing material composition comprising an acyl acidic amino acid or a salt thereof, an amphoteric surfactant, and hydroxypropyl starch phosphate, wherein the cleansing material composition has a pH of 5.3 or less.

(2) The cleansing material composition according to (1), wherein a content of the hydroxypropyl starch phosphate is 0.1 to 20% by mass.

(3) The cleansing material composition according to (1) or (2), wherein a content (A) of the acyl acidic amino acid or the salt thereof, a content (B) of the amphoteric surfactant, and a content (C) of the hydroxypropyl starch phosphate satisfy a relationship represented by the following formula 1 on a mass basis

((A)+(B))/(C)=2.5 to 20. (formula 1):

(4) The cleansing material composition according to any one of (1) to (3), wherein a ratio (AB) between a content (A) of the acyl acidic amino acid or the salt thereof and a content (B) of the amphoteric surfactant is in a range of 0.3 to 3 on a mass basis.

(5) The cleansing material composition according to any one of (1) to (4), wherein a total of a content (A) of the acyl acidic amino acid or the salt thereof and a content (B) of the amphoteric surfactant is 5% by mass or more.

(6) The cleansing material composition according to any one of (1) to (5), wherein a content (A) of the acyl acidic amino acid or the salt thereof is 1 to 25% by mass.

(7) The cleansing material composition according to any one of (1) to (6), further comprising a polymer.

(8) The cleansing material composition according to any one of (1) to (7), further comprising an oil agent.

(9) The cleansing material composition according to (8), wherein the oil agent includes solid oil or semi-solid oil.

(10) The cleansing material composition according to any one of (1) to (9), further comprising a polyhydric alcohol.

(11) The cleansing material composition according to (10), wherein a content of the polyhydric alcohol is 1 to 40% by mass.

(12) The cleansing material composition according to (10) or (11), wherein the polyhydric alcohol contains glycerin or sorbitol.

(13) The cleansing material composition according to any one of (1) to (12), wherein the amphoteric surfactant includes a sultaine-type surfactant.

(14) The cleansing material composition according to any one of (1) to (13), wherein a viscosity at 25° C. is 3,500 mPa·s or more.

(15) The cleansing material composition according to any one of (1) to (14), which is used for face washing.

(16) The cleansing material composition according to any one of (1) to (15), which is in a gel-form.

(17) The cleansing material composition according to any one of (1) to (16), wherein the acyl acidic amino acid or the salt thereof is acyl glutamic acid or a salt thereof.

The present invention provides a cleansing material composition using an acyl acidic amino acid, the cleansing material composition having a viscosity suitable for tube-filled use without impairing foam performance such as foam volume and foamability.

### DETAILED DESCRIPTION OF THE PREFERRED EMBODIMENTS

The cleansing material composition according to an embodiment of the present invention contains an acyl acidic amino acid or a salt thereof and an amphoteric surfactant, and has a pH of 5.3 or less. In addition, the cleansing material composition also contains hydroxypropyl starch phosphate. By employing such a configuration, it is possible to obtain a viscosity suitable for tube-filled use without impairing foam performance.

### Acyl Acidic Amino Acid or Salt Thereof

The composition of the invention of the present application contains an acyl acidic amino acid or a salt thereof. The “acidic amino acid” in the acyl acidic amino acid or the salt thereof is not particularly limited, and examples thereof include glutamic acid and aspartic acid, and glutamic acid is preferable.

As an acyl acidic amino acid such as glutamic acid or a salt thereof, any of the optical isomers of L-form, D-form, and DL-form can be used.

Examples of the “acyl” in the acyl acidic amino acid or the salt thereof include an acyl group having 10 to 26 and preferably 12 to 18 carbon atoms. Specific examples include an octanoyl group, a decanoyl group, a lauroyl group, a myristoyl group, a palmitoyl group, a stearoyl group, a behenyl group, an oleoyl group, and a coconut oil fatty acid acyl group, and a coconut oil fatty acid acyl group is preferable.

The “salt” in the acyl acidic amino acid or the salt thereof is not particularly limited, and examples thereof include salts of alkali metals such as sodium and potassium, salts of alkaline earth metals such as calcium and magnesium, inorganic salts containing e.g. aluminum and zinc, salts of organic amines such as ammonia, monoethanolamine, diethanolamine, and triethanolamine, and organic salts of e.g. basic amino acids such as arginine and lysine. An alkali metal salt is preferable, and a sodium salt is more preferable.

The acyl acidic amino acid or the salt thereof is preferably N-lauroyl-L-sodium glutamate, N-lauroyl-L-potassium glutamate, N-lauroyl-L-disodium glutamate, N-lauroyl-L-dipotassium glutamate, N-lauroyl-L-glutamic acid triethanolamine, N-coconut oil fatty acid acyl-L-sodium glutamate, N-coconut oil fatty acid acyl-L-potassium glutamate, N-coconut oil fatty acid acyl-L-disodium glutamate, N-coconut oil fatty acid acyl-L-dipotassium glutamate, or N-coconut oil fatty acid acyl-L-glutamic acid triethanolamine. These may be used alone or in combination.

### Amphoteric Surfactant

The composition of the invention of the present application contains an amphoteric surfactant. The amphoteric surfactant is at least one selected from the group consisting of lauryl hydroxysultaine, coco-hydroxysultaine, lauramide hydroxysultaine, lauramidopropyl hydroxysultaine, cocamidopropyl hydroxysultaine, palm kernel fatty acid amidopropyl hydroxysultaine, lauryldimethylaminoacetic acid betaine, palm oil alkyl betaine, stearyldimethylaminoacetic acid betaine, stearyl dihydroxyethyl betaine, lauramidopropyl betaine, cocoamidopropyl betaine, palm kernel fatty acid amidopropyl betaine, ricinoleic acid amidopropyl betaine, N-coconut oil fatty acid acyl-N-carboxyethyl-N-hydroxyethylethylenediamine salt, palm kernel oil fatty acid acyl-N-carboxyethyl-N-hydroxyethylethylenediamine salt, 2-alkyl-N-carboxymethyl-N-hydroxyethylimidazolium betaine, undecyl-N-carboxymethylimidazolinium betaine, alkyldiaminoethylglycine hydrochloride solution, bis(stearyl-N-hydroxyethylimidazoline)chloroacetic acid complex, sodium cocoamphoacetate, disodium cocoamphodiacetate, sodium cocoamphopropionate, cocobetaine, sodium lauroamphoacetate, and stearyl betaine.

Preferable amphoteric surfactants are sodium lauroamphoacetate, sodium cocoamphoacetate, lauramidopropyl betaine, and sulfonic acid group-containing amphoteric surfactants. Preferable examples of the sulfonic acid group-containing amphoteric surfactants include sultaine-type amphoteric surfactants. Examples of the sultaine-type amphoteric surfactants include lauryl hydroxysultaine, coco-hydroxysultaine, lauramidopropyl hydroxysultaine, cocamidopropyl hydroxysultaine, and palm kernel fatty acid amidopropyl hydroxysultaine.

A more preferable amphoteric surfactant is lauramidopropyl hydroxysultaine.

### Additional Surfactants

The composition of the present invention may further contain an anionic surfactant and a nonionic surfactant in addition to the above-described active agents.

Examples of the anionic surfactants include alkyl sulfates, alkyl phosphates, polyoxyethylene alkyl ether sulfates, polyoxyethylene alkyl phenyl ether sulfates, polyoxyethylene alkyl carboxylates, alkyl phenyl ether sulfonates, salts of alkylsulfosuccinic acid and derivatives thereof, salts of alkyl sarcosine and derivatives thereof, N-alkyl-N-methyl-β-alanine salts, polyoxyethylene coconut oil fatty acid monoethanolamide sulfates, polyoxyethylene alkyl ether phosphates, long-chain fatty acid ethyl ester sulfonates, higher fatty acid salts, and alkyl ether carboxylic acid.

The composition of the present invention can be in an embodiment essentially containing no anionic surfactants.

Examples of nonionic surfactants can include fatty acid monoethanolamide, fatty acid diethanolamide, polyoxyethylene fatty acid monoethanolamide, alkyl glycoside derivatives, and sugar alcohol hydroxy aliphatic ether derivatives.

The composition of the present invention can be in an embodiment essentially containing no nonionic surfactants.

The composition of the present invention can be thickened by entanglement between the surfactants and the polymer and achieves an advantage that the concentration of the surfactants blended can be kept low, making it unnecessary to perform a complicated crystallization operation. Therefore, the total amount of the surfactants in the composition can be, for example, 30% by mass or less, preferably 25% by mass or less, more preferably 24% by mass or less, further preferably 22% by mass or less, and further preferably 20% by mass or less. Here, the “total amount of surfactants” means the total amount of the acyl acidic amino acid or the salt thereof, the amphoteric surfactant, and, if contained, the additional anionic surfactant and nonionic surfactant. The total amount of the surfactants can be, for example, 5% by mass or more, preferably 10% by mass or more, and more preferably 12% by mass or more, and is preferably 5 to 30% by mass, more preferably 5 to 25% by mass, and more preferably 5 to 20% by mass.

The total (A+B) of the content (A) of the acyl acidic amino acid or the salt thereof and the content (B) of the amphoteric surfactant in the composition according to the present embodiment can be, for example, 30% by mass or less, preferably 25% by mass or less, more preferably 24% by mass or less, further preferably 22% by mass or less, and further preferably 20% by mass or less. In addition, the total can be, for example, 5% by mass or more, preferably 10% by mass or more, and more preferably 12% by mass or more, and is preferably 5 to 30% by mass, more preferably 5 to 25% by mass, and more preferably 5 to 20% by mass.

The mass ratio (A/B) between the content (A) of the acyl acidic amino acid or the salt thereof and the content (B) of the amphoteric surfactant is preferably 0.3 to 3, more preferably 0.5 to 3, and further preferably 0.7 to 3.

When the mass ratio (A/B) is 0.3 or more, the effect of improving the rapid foaming property during foaming is obtained. Meanwhile, when the mass ratio (A/B) is 3 or less, the effect of improving the foam density is obtained.

The content (A) of the acyl acidic amino acid or the salt thereof in the composition is, for example, 1 to 25% by mass, preferably 5 to 20% by mass, more preferably 5 to 15% by mass, and further preferably 5 to 12% by mass.

When the content (A) is 1% by mass or more, the foam quality improvement effect that large bubbles are easily formed during foaming is obtained. Meanwhile, when the content (A) is 25% by mass or less, the effect of improving the rapid foaming property is obtained.

### pH Adjuster

The pH of the composition according to the present embodiment may be 5.3 or less, and more preferably 4 to 5.3. When the pH is 5.3 or less, it becomes easy to obtain a composition having a high viscosity.

The pH can be adjusted by, for example, adding a pH adjuster. A preferable pH adjuster includes, for example, an citric acid aqueous solution.

### Hydroxypropyl Starch Phosphate

The composition according to the present embodiment contains hydroxypropyl starch phosphate as described above.

When hydroxypropyl starch phosphate is added, the effect of thickening the composition is obtained. The content (C) of hydroxypropyl starch phosphate is, for example, 0.1 to 20% by mass, preferably 1 to 10% by mass, and more preferably 1 to 8% by mass. When the content of hydroxypropyl starch phosphate is 0.1% by mass or more, the effect of improving storage stability can be obtained. Meanwhile, when the content of hydroxypropyl starch phosphate is 20% by mass or less, the effect of stabilizing a prescription viscosity with time is obtained.

Preferably, the content (A) of the acyl acidic amino acid or the salt thereof, the content (B) of the amphoteric surfactant, and the content (C) of the hydroxypropyl starch phosphate satisfy a relationship represented by the following formula 1 on a mass basis

((A)+(B))/(C)=2.5 to 20. (formula 1):

When the value of ((A)+(B))/(C) is 2.5 or more, an abundant foam volume can be obtained, and when the value of ((A)+(B))/(C) is 20 or less, an effect of imparting viscosity excellent in usability can be obtained. The value is preferably 15 or less, and preferably 10 or less.

### Additional Polymers

The composition according to the present embodiment may contain an additional polymer. Examples of the additional polymer include acrylates copolymers (alkyl acrylates (C1-4), alkyl methacrylates (C1-4), copolymers of two or more components of acrylic acid and methacrylic acid), highly polymerized polyethylene glycol (for example, polyethylene glycol having a polymerization degree of 5000 to 50000), (acrylates/vinyl neodecanoate) cross polymers, polyethylene glycol distearate, Ceteares-60 Myristyl Glycol, guar gum, xanthan gum, starch, ethyl cellulose, methyl hydroxypropyl starch, carboxymethylcellulose, carboxyvinyl polymers, bentonite, and hectorite.

Examples of preferable polymers include acrylates copolymers, highly polymerized polyethylene glycol, polyethylene glycol distearate, and Ceteares-60 Myristyl Glycol.

The content of the additional polymer is, for example, 5% by mass or less, preferably 3% by mass or less, and more preferably 1.5% by mass or less, and, for example, 0.01% by mass or more, preferably 0.05% by mass or more, and more preferably 0.1% by mass or more.

### Oil Agent

The composition according to the present embodiment preferably contains an oil agent. The oil agent is not particularly limited, and it is possible to use a known oil agent.

The oil agent used is preferably solid oil or semi-solid oil.

Examples of the oil agent include animal- and plant-derived oils such as vaseline, squalane, jojoba oil, macadamia nut oil, camellia oil, corn oil, olive oil, rapeseed oil, coconut oil, palm oil, castor oil, hardened castor oil, beeswax, liquid lanolin, liquid paraffin, squalene, mink oil, castor oil, egg yolk oil, camellia oil, soybean oil, linseed oil, avocado oil, lanolin, safflower oil, sunflower oil, rosemary oil, mineral oils, chain polysiloxanes such as dimethylpolysiloxane, cyclic polysiloxanes such as octamethylcyclotetrasiloxane, and amino-modified silicone oils. The animal- and plant-derived oil or mineral oil usable is, for example, a linear type, ester type, or triglyceride type hydrocarbon-based one.

Examples of preferable oil agents include vaseline, squalane, jojoba oil, and macadamia nut oil.

The content of the oil agent is, for example, 1 to 30% by mass, preferably 3 to 20% by mass, and more preferably 5 to 15% by mass.

When the content of the oil agent is 1% by mass or more, an effect of improving touch is obtained. Meanwhile, when the content of the oil agent is 30% by mass or less, a thickening effect is obtained.

### Polyhydric Alcohol

The composition according to the present embodiment may contain a polyhydric alcohol. When a polyhydric alcohol is contained, it becomes easy to prepare a composition having a viscosity which allows easy discharge without dripping from the tube.

Examples of the polyhydric alcohol include glycerin, sorbitol, butylene glycol, dipropylene glycol, propanediol, propylene glycol, isoprene glycol, and diglycerin.

Examples of preferable polyhydric alcohols include glycerin and sorbitol.

The content of the polyhydric alcohol is, for example, 1 to 40% by mass, preferably 2 to 40% by mass, more preferably 3 to 30% by mass, and further preferably 3.5 to 25% by mass.

### Water

The composition according to the present embodiment preferably contains water as a solvent. The water is not particularly limited as long as it is pure enough to be used for a cleansing agent or a cleansing material. Specifically, ion exchange water, well water, natural water, ground water, city water, hard water, soft water, and the like can be used.

### Additional Components

In addition to the above-described components, the composition according to the present embodiment can be appropriately blended with various components used in a normal cleansing material composition. Examples of such components include chelating agents, preservatives, fragrances, ultraviolet absorbers, humectants, amino acids, bioactive ingredients, antioxidants, pearlizing agents, anti-inflammatory agents, antibacterial agents, scrub agents, and antiperspirants.

The composition according to the present embodiment is supplied to the user in the state where the inverse stand tube is filled with the composition. The composition according to the present embodiment has a viscosity which allows easy discharge from the inverse stand tube, and can be used suitably as a gel face-washing composition such as face-washing foam.

In the composition of the present invention, the viscosity at 25° C. is 3,500 mPa·s or more, preferably 4,000 mPa·s or more, more preferably 5,000 mPa·s or more, further preferably 10,000 mPa·s or more, and 300,000 mPa·s or more. Note that the viscosity at 25° C. is calculated as measured values obtained by measuring with a B-type rotational viscometer with rotor No. 1 or No. 2 at a rotational speed of 6 rpm, 12 rpm, and 60 rpm at 25° C.

### Production Method

The composition according to the present embodiment can be obtained by mixing with water, a surfactant, hydroxypropyl starch phosphate, and an oil agent as well as an additional additive as needed. Specifically, all the components are heated and stirred for uniform mixing, and then the temperature is lowered to 25° C. to obtain a composition.

Other features of the invention will become apparent in the course of the following descriptions of exemplary embodiments which are given for illustration of the invention and are not intended to be limiting thereof.

### EXAMPLES

In the following examination example, the viscosity was measured by the following method.

### Viscosity Measurement

The viscosity (mPa·s) of each composition obtained was measured as the original viscosity. For measurement of the viscosity, a B-type viscometer manufactured by Toyo Seiki Seisaku-sho, Ltd. was used. Specifically, the sample was filled in a glass vial container and left in a room set at 25° C., and then the measurement was performed. The measurement conditions (rotor No, rotation speed, measurement time) were appropriately adjusted according to the maximum measurable viscosity, and the measurement was performed.

For each prepared cleansing agent composition of Examples and Comparative Examples, the foam volume and foamability were evaluated as follows.

### Measurement of Foam Volume

Method: About 1 g of sample was placed on the palm and foamed using tap water with a constant water flow, and the foam volume was evaluated according to the following evaluation criteria.

Evaluation Criteria:

A: foamed

B: somewhat foamed

C: hardly foamed

### Foamability

Method: About 1 g of sample was placed on the palm and foamed using tap water with a constant water flow, and the foamability was evaluated according to the following evaluation criteria.

Evaluation Criteria:

A: the composition is easily soluble during foaming

B: the composition is somewhat soluble during foaming

C: the composition is hard to dissolve during foaming

### Examination Example 1: Examination in Prescription of Face-Washing Foam

The components were mixed in the compositions presented in the table to obtain compositions according to Examples and Comparative Examples.

The table also presents the evaluation results for the obtained compositions. The numerical values described in the table indicate the solid content of active ingredient.

TABLE 1

| | | | Example | | | | | |
|---|---|---|---|---|---|---|---|---|
| | | Component Name | 1 | 2 | 3 | 4 | 5 | 6 |
| A | Amino | Disodium N-Cocoyl-L-glutamate | 8.5 | 8.5 | 8.5 | 8.5 | 8.5 | 8.5 |
| — | Acid-Based | Sodium N-Cocoylglycinate | | | | | | |
| | Surfactant | | | | | | | |
| B | Amphoteric | Lauramidopropyl Hydroxysultaine | 4.3 | 8.5 | 3 | 12 | 4.3 | 4.3 |
| B | Surfactant | Sodium Cocoamphoacetate | 4.3 | | | | 4.3 | 4.3 |
| — | Surfactant | Decyl Glucoside | | | | | | |
| C | | Hydroxypropyl Starch Phosphate | 4 | 4 | 4 | 4 | 2 | 8 |
| | Polyhydric | Sorbitol | 5 | 5 | 5 | 5 | 5 | 5 |
| | Alcohol | | | | | | | |
| | Polymer | PEG 14M | 0.5 | 0.5 | 0.5 | 0.5 | 0.5 | 0.5 |
| | | Acrylates Copolymer | 1 | 1 | 1 | 1 | 1 | 1 |
| | pH Adjuster | 20% Citric Acid Aqueous Solution | q.s. | q.s. | q.s. | q.s. | q.s. | q.s. |
| | | Water | Balance | Balance | Balance | Balance | Balance | Balance |
| | | Total | 100 | 100 | 100 | 100 | 100 | 100 |
| | | pH | 5.3 | 4.9 | 5.3 | 5.0 | 5.1 | 5.2 |
| | | Viscosity | 91,200 | >180,000 | 13,640 | >180,000 | 37,020 | 7,500 |
| | | Foam Volume | A | A | A | A | A | A |
| | | Foamability | A | A | A | A | A | A |
| | | Total Content of Surfactants | 17.1 | 17 | 11.5 | 20.5 | 17.1 | 17.1 |
| | | Total Content (B) of Amphoteric | 8.6 | 8.5 | 3 | 12 | 8.6 | 8.6 |
| | | Surfactant | | | | | | |
| | | (A)/(B) | 1.0 | 1.0 | 2.8 | 0.7 | 1.0 | 1.0 |
| | | (A + B)/C | 4.3 | 4.3 | 2.9 | 5.1 | 8.6 | 2.1 |

| | | | Comparative Example | | | | |
|---|---|---|---|---|---|---|---|
| | | Component Name | 1 | 2 | 3 | 4 | 5 |
| A | Amino | Disodium N-Cocoyl-L-glutamate | 8.5 | 8.5 | 8.5 | 8.5 | |
| — | Acid-Based | Sodium N-Cocoylglycinate | | | | | 8.5 |
| | Surfactant | | | | | | |
| B | Amphoteric | Lauramidopropyl Hydroxysultaine | 4.3 | | 4.3 | 4.3 | 4.3 |
| B | Surfactant | Sodium Cocoamphoacetate | 4.3 | | 4.3 | 4.3 | 4.3 |
| — | Surfactant | Decyl Glucoside | | 8.5 | | | |
| C | | Hydroxypropyl Starch Phosphate | | 4 | 4 | 4 | 4 |
| | Polyhydric | Sorbitol | 5 | 5 | 5 | 5 | 5 |
| | Alcohol | | | | | | |
| | Polymer | PEG 14M | 0.5 | 0.5 | 0.5 | 0.5 | 0.5 |
| | | Acrylates Copolymer | 1 | 1 | 1 | 1 | 1 |
| | pH Adjuster | 20% Citric Acid Aqueous Solution | q.s. | q.s. | q.s. | q.s. | q.s. |
| | | Water | Balance | Balance | Balance | Balance | Balance |
| | | Total | 100 | 100 | 100 | 100 | 100 |
| | | pH | 5.3 | 4.8 | 6.0 | 6.4 | 5.3 |
| | | Viscosity | 3,330 | 1,512 | 3,340 | 808 | >180,000 |
| | | Foam Volume | A | A | A | B | C |
| | | Foamability | A | A | A | A | C |
| | | Total Content of Surfactants | 17.1 | 17 | 17.1 | 17.1 | 17.1 |
| | | Total Content (B) of Amphoteric | 8.6 | 0 | 8.6 | 8.6 | 8.6 |
| | | Surfactant | | | | | |
| | | (A)/(B) | — | — | 1.0 | 1.0 | — |
| | | (A + B)/C | — | — | 4.3 | 4.3 | — |

Comparative Example 5 using an acyl neutral amino acid in place of the acyl acidic amino acid was inferior in terms of foam volume and foamability.

Comparative Example 2 using decyl glucoside (nonionic surfactant) in place of the amphoteric surfactant failed to obtain sufficient viscosity.

The composition according to Examples containing hydroxypropyl starch phosphate had higher viscosity as well as better usability than those of Comparative Example 1 lacking hydroxypropyl starch phosphate.

The compositions according to Comparative Examples 3 and 4 having a pH exceeding 5.3 failed to obtain sufficient viscosity. In the case of use after being filled in an inverse stand tube, these compositions easily dripped when the cap was opened or when the composition was transferred onto the palm. Therefore, it was impossible to say that these compositions had an appropriate viscosity as a composition for an inverse stand tube.

On the other hand, the compositions according to Examples having a pH of 5.3 or less had a viscosity higher than those of Comparative Examples. In addition, none of Examples was too viscous to be discharged from the inverse stand tube.

TABLE 2

| | | Component Name | Example 7 | Example 8 |
|---|---|---|---|---|
| A | Amino Acid-Based | Disodium | 7.5 | 7.5 |
| | Surfactant | N-Cocoyl-L-glutamate | | |
| B | Amphoteric | Lauramidopropyl | 7.5 | 7.5 |
| | Surfactant | Hydroxysultaine | | |
| C | | Hydroxypropyl Starch | 5 | 5 |
| | | Phosphate | | |
| | Polyhydric | Sorbitol | 20 | |
| | Alcohol | Glycerin | | 10 |
| | Oil Agent | Vaseline | | 10 |
| | pH Adjuster | 20% Citric Acid | q. s. | q. s. |
| | | Aqueous Solution | | |
| | | Water | Balance | Balance |
| | | Total | 100 | 100 |
| | | pH | 5.1 | 5.1 |
| | | Viscosity | 42,000 | 27,380 |
| | | Total Content of | 15 | 15 |
| | | Surfactants | | |
| | | Total Content (B) of | 7.5 | 7.5 |
| | | Amphoteric Surfactant | | |
| | | (A)/(B) | 1 | 1 |
| | | (A + B)/C | 3 | 3 |

Example 7 using sorbitol as the polyhydric alcohol exhibited good viscosity. Example 8 using glycerin as the polyhydric alcohol in place of sorbitol and vaseline as the oil agent also exhibited good viscosity.

TABLE 3

| | | | Example | | | | | |
|---|---|---|---|---|---|---|---|---|
| | | Component Name | 1 | 9 | 10 | 11 | 12 | 13 |
| A | Amino | Disodium N-Cocoyl-L-glutamate | 8.5 | 8.5 | 6.7 | 10 | 10 | 10.7 |
| | Acid-Based | | | | | | | |
| | Surfactant | | | | | | | |
| B | (B) Amphoteric | Lauramidopropyl Hydroxysultaine | 4.3 | 4.3 | 2.7 | 4 | | 2.7 |
| B | Surfactant | Sodium Cocoamphoacetate | 4.3 | | | | | |
| B | Component | Sodium Lauroamphoacetate | | 4.3 | 2.7 | 4 | 4 | |
| B | | Lauramidopropyl Betaine | | | | | 4 | 2.7 |
| C | | Hydroxypropyl Starch Phosphate | 4 | 4 | 4 | 3 | 3 | 3 |
| | Oil Agent | Vaseline | | | 10 | 10 | 10 | 10 |
| | | Macadamia Nut Oil | | 10 | | | | |
| | Polyhydric | Glycerin | | | 10 | 10 | 10 | |
| | Alcohol | Sorbitol | 5 | 5 | 10 | 10 | 10 | 10 |
| | Polymer | PEG 14M | 0.5 | 0.2 | 0.2 | | | 0.2 |
| | | Acrylates Copolymer | 1 | 1 | 1 | 0.6 | 0.6 | 0.6 |
| | pH Adjuster | 20% Citric Acid Aqueous Solution | q.s. | q.s. | q.s. | q.s. | q.s. | q.s. |
| | | Water | Balance | Balance | Balance | Balance | Balance | Balance |
| | | Total | 100 | 100 | 100 | 107 | 106 | 100 |
| | | pH | 5.1 | 5.1 | 5.1 | 5.1 | 5.1 | 5.1 |
| | | Viscosity | >180,000 | >180,000 | >180,000 | 78,600 | 34,210 | >180,000 |
| | | Total Content of Surfactants | 17 | 17 | 12 | 18 | 18 | 16 |
| | | Total Content (B) of Amphoteric | 8.5 | 8.5 | 5.3 | 8 | 8 | 5.3 |
| | | Surfactant | | | | | | |
| | | (A)/(B) | 1 | 1 | 1.3 | 1.3 | 1.3 | 2 |
| | | (A + B)/C | 4.3 | 4.3 | 3 | 6 | 6 | 5.3 |

Examples 1, 9 to 11, and 13 using a sultaine-type amphoteric surfactant as the amphoteric surfactant had a higher viscosity than that of Example 12 not using a sultaine-type amphoteric surfactant.

The suppliers of the main components used and their trade names are as follows.

| Component Name | Trade name | Supplier |
|---|---|---|
| Disodium | AMISOFT ECS-22W | Ajinomoto Co., Inc. |
| N-Cocoyl-L-glutamate | | |
| (i.e N-Coconut Oil | | |
| Fatty Acid | | |
| Acyl-L-Disodium | | |
| Glutamate) | | |
| Lauramidopropyl | SOFTAZOLINE LSB | Kawaken Fine |
| Hydroxysultaine | | Chemicals Co., Ltd. |
| Sodium | SOFTAZOLINE CH | Kawaken Fine |
| Cocoamphoacetate | | Chemicals Co., Ltd. |
| Vaseline | Vaseline (Sun White) | Nikko Rica |
| | | Corporation |
| Macadamia Nut Oil | Macadamia Nut Oil | Nikko Chemicals Co., |
| | | Ltd. |
| Glycerin | Concentrated | Kao Corporation |
| | Glycerin | |
| Sorbitol | Sorbitol | Kao Corporation |
| Acrylates Copolymer | Aqua SF-1 | Lubrizol |
| PEG-14M | POLYOX WSRN | Dow Chemical |
| | 3000 AMERCHOL | |
| Hydroxypropyl | STRUCTURE XL | Akzo Nobel |
| Starch Phosphate | | |

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A cleansing material composition, comprising:
at least one acyl acidic amino acid or a salt thereof;
at least one amphoteric surfactant; and
at least one hydroxypropyl starch phosphate,
wherein said cleansing material composition has a pH of 5.3 or less.

2. The cleansing material composition according to claim 1, wherein a content of said at least one hydroxypropyl starch phosphate is 0.1 to 20% by mass.

3. The cleansing material composition according to claim 1, wherein a content (A) of said at least one acyl acidic amino acid or salt thereof, a content (B) of said at least one amphoteric surfactant, and a content (C) of said at least one hydroxypropyl starch phosphate satisfy a relationship represented by the following formula 1 on a mass basis
((A)+(B))/(C)=2.5 to 20. formula 1:

4. The cleansing material composition according to claim 1, wherein a ratio (A/B) between a content (A) of said at least one acyl acidic amino acid or salt thereof and a content (B) of said at least one amphoteric surfactant is in a range of 0.3 to 3 on a mass basis.

5. The cleansing material composition according to claim 1, wherein a total of a content (A) of said at least one acyl acidic amino acid or salt thereof and a content (B) of said at least one amphoteric surfactant is 5% by mass or more.

6. The cleansing material composition according to claim 1, wherein a content (A) of said at least one acyl acidic amino acid or salt thereof is 1 to 25% by mass.

7. The cleansing material composition according to claim 1, further comprising at least one polymer.

8. The cleansing material composition according to claim 1, further comprising at least one oil agent.

9. The cleansing material composition according to claim 8, wherein said at least one oil agent comprises a solid oil or a semi-solid oil.

10. The cleansing material composition according to claim 1, further comprising at least one polyhydric alcohol.

11. The cleansing material composition according to claim 10, wherein a content of said at least one polyhydric alcohol is 1 to 40% by mass.

12. The cleansing material composition according to claim 10, wherein said at least one polyhydric alcohol comprises glycerin or sorbitol.

13. The cleansing material composition according to claim 1, wherein said at least one amphoteric surfactant comprises a sultaine-type surfactant.

14. The cleansing material composition according to claim 1, which has a viscosity at 25° C. is 3,500 mPa·s or more.

15. The cleansing material composition according to claim 1, wherein said at least one acyl acidic amino acid or salt thereof is acyl glutamic acid or a salt thereof.

16. A method of washing a face, comprising applying a cleansing material composition according to claim 1, to said face.
